(19) United States
(12) Patent Application Publication
(10) Pub. No.: US 2007/0016120 A1
(43) Pub. Date: Jan. 18, 2007

(54) Posture band and method of improving posture

(76) Inventors: Miguel J. Latronica, Chicago, IL (US); Jeanie H. Pollack, Chicago, IL (US)

Correspondence Address: PERRY HOFFMAN & ASSOCIATES P.C., PO BOX 1649, DEERFIELD, IL, 60015, US

(21) Appl. No.: 11/485,121

(22) Filed: Jul. 12, 2006

Related U.S. Application Data
(60) Provisional application No. 60/698,753, filed on Jul. 13, 2005.

(51) Int. Cl. A61F 5/00

(52) U.S. Cl. 602/19; 602/20

(57) ABSTRACT

A posture band including an elastomeric body which easily positions on a user and creates a variety of tension strengths along specific sections of the band training the user to achieve and maintain correct posture and spinal alignment while at work, rest, or play. A positioning mechanism is coupled to the body for facilitating the proper alignment and tensioning of the body on a user wherein the positioning mechanism rests on the back of the neck of the user training the user to roll his/her shoulders back bringing the shoulder blades together and holding up his/her head. A covering manufactured from a wearable material incorporates a substantial portion of the body therein, and provides a more comfortable feel for the user and is also fashionable allowing the user to wear the band on top of as well as underneath their clothing.

### CROSS-REFERENCE TO RELATED APPLICATION

This application claims priority pursuant to 35 USC 119(e) to U.S. Provisional Application No. 60/698,753 filed on Jul. 13, 2005 which is incorporated herein by reference in its entirety.

### BACKGROUND OF THE INVENTION

1. Field of the Invention

The present invention relates to posture training devices, and more particularly to a posture band which easily positions on a user and creates a variety of tension strengths along specific sections of the band training the user to achieve and maintain correct posture and spinal alignment while at work, rest, or play. The invention also relates to a method for improving the user's posture through the specifically directed tensioning of the posture band of the present invention.

2. Background of the Invention

Poor posture is a serious problem afflicting millions of Americans and contributes to numerous health problems such as back aches, hunched backs, tension headaches, and overall poor health and inflexibility of the spine. Poor posture is so wide spread that solutions to this problem have been attempted by many different sources, including medical companies selling therapeutic support devices and fitness companies selling posture training devices.

The therapeutic support devices sold in the marketplace attempt to relieve some of the painful effects of poor posture, such as an aching back for example, by supporting and somewhat immobilizing specific parts of the back, neck or shoulders, in an effort to relieve some of the user's discomfort. These therapeutic support devices are not effective in training the user to achieve and maintain proper posture because the devices simply restrict a user from moving specific body parts in order to relieve discomfort, and do not retrain muscles, or the user, to maintain proper posture. Further, excessive use of an upper body muscle support device could result in the eventual weakening of some upper body muscles from their non-use while wearing the device, potentially worsening the user's posture.

The known posture training devices focus mainly on helping the user achieve a proper position to improve the user's performance in a specific activity, such as golf for example. These devices are not practical or effective in training the user to achieve and maintain proper posture while working or resting, because they are cumbersome or impractical to wear, sometimes too heavy to wear for any length of time, not easy to adjust or they simply are not intended to be worn beyond participation in the specific activity or training session. Additionally, many of the known devices contain complicated signal and feedback transmitters and circuitry to provide feedback relating to the user's posture to the user, or a third party, rather than training the user to recognize the feel of their own body as it achieves and maintains proper posture, and create muscle memory in the user when proper posture is achieved.

### SUMMARY OF THE INVENTION

The present invention addresses shortcomings of the prior art to provide a posture band which easily positions on a user and creates a variety of tension strengths along the band training the user to achieve and maintain correct posture and spinal alignment. The posture band of the present invention is comfortable to wear and can be worn continuously as a user transitions from resting to playing and/or working because the band can be quickly and simply loosened or tightened while worn by the user, thus adjusting the specific amount of tension exhibited by the band when worn during various activities.

In one embodiment of the invention, the posture band includes an elastomeric body and a positioning mechanism coupled to the body for facilitating the proper alignment and tensioning of the body on the user. The positioning mechanism rests on the back of the neck of the user training the user to roll his/her shoulders back bringing the shoulder blades together and holding up his/her head.

In another embodiment of the invention, the posture band includes one or more recesses cut into a width of the body and/or a width of the positioning mechanism for facilitating the proper positioning of the band on the user.

In another embodiment, the positioning mechanism includes a first layer and a second layer of elastomeric material coupled together. The first layer may have a longer length than the second layer providing a varying tension along the positioning mechanism as it is stretched while in use. A portion of either the first or second layers of the positioning mechanism may also have a wider width than the other of the first or second layers.

In yet another embodiment, a semi-rigid insert adjacent the body is included to enhance the effect of the positioning mechanism. In another embodiment, a small foam insert or the like adjacent the positioning mechanism is included as a tactile reminder to the user as to the proper positioning of his/her head. The body also includes first and second ends and a buckle mechanism coupled to both ends to facilitate the correct fit of the body to the user and provide a means for adjusting the tensioning of the body while in use from either of the first or second ends.

In another embodiment of the invention, the posture band includes a covering capable of incorporating a substantial portion of the body therein. The semi-rigid insert is also at least partially contained within the covering.

In another embodiment, a method for improving a user's posture includes providing a posture band having a body made of elastomeric material and a positioning mechanism coupled to the body. The body of the band is wrapped around the back of the neck, over both shoulders and around the back of the user, and the band is adjusted so the positioning mechanism rests on the back of the neck of the user training the user to rotate his/her shoulders back bringing the shoulder blades together, holding up his/her head and thus improving the user's posture.

### BRIEF DESCRIPTION OF THE DRAWINGS

For the purpose of facilitating an understanding of the inventions, the accompanying drawings and description illustrate a preferred embodiment thereof, from which the inventions, structure, construction and operation, and many related advantages may be readily understood and appreciated.

FIG. 1 is a perspective view of a posture band of the present invention illustrating a body including a partial second layer and a positioning mechanism having a first and second layer;

FIG. 2 is a perspective view of an embodiment of the posture band in use illustrating a buckle in open and closed positions;

FIG. 3 is another perspective view of the posture band embodiment of FIG. 2 illustrating the positioning mechanism in use and the semi-rigid insert;

FIG. 4 is a perspective view of an embodiment of the posture band in use illustrating a loop of the elastomeric body protruding from a covering for easy adjustment of the posture band while in use;

FIG. 5 is a perspective view of the buckle illustrating a first side released from a second side;

FIG. 6 is a perspective view of an embodiment of the posture band in use illustrating a foam insert or the like coupled to the positioning mechanism;

FIG. 7 is a perspective view of the posture band embodiment illustrating use with a portable music player; and

FIG. 8 is a perspective view of the posture band embodiment illustrating use with a flap cover structure covering the buckle, ends of the posture band body and for securing personal accessories and effects.

### DETAILED DESCRIPTION OF THE PREFERRED EMBODIMENTS

The following description is provided to enable those skilled in the art to make and use the described embodiments set forth in the best modes contemplated for carrying out the invention. Various modifications, however, will remain readily apparent to those skilled in the art. Any and all such modifications, equivalents, and alternatives are intended to fall within the spirit and scope of the present invention.

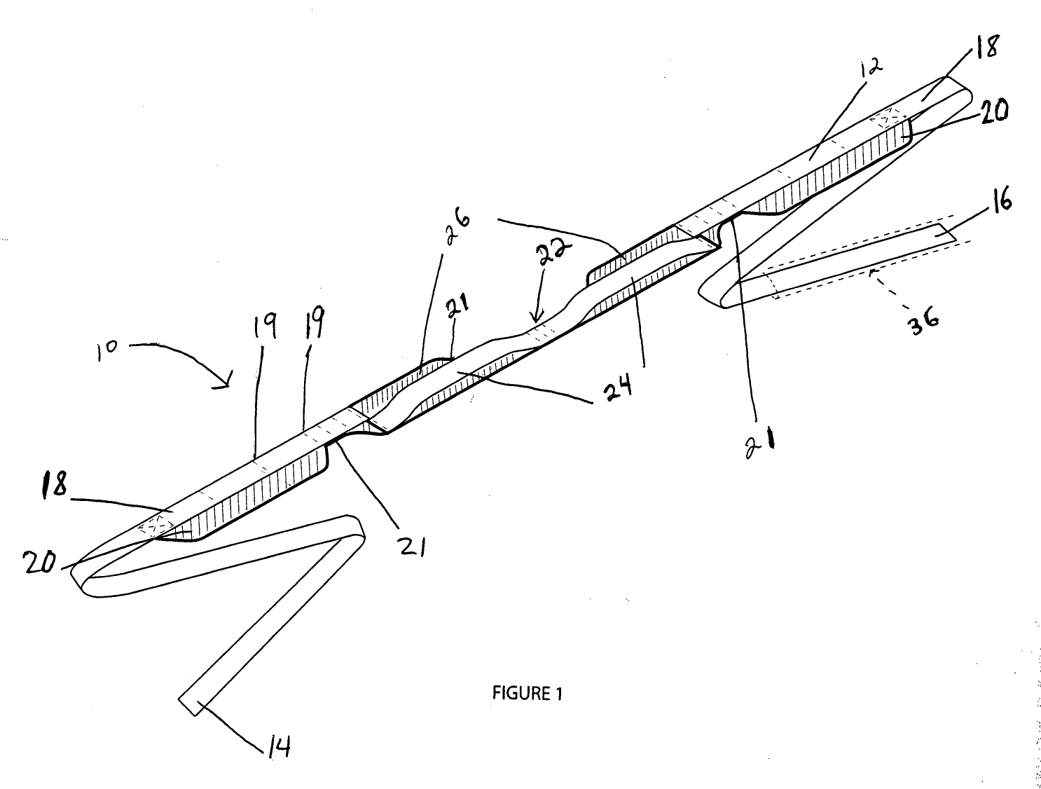

A posture band 10, as seen in FIG. 1, includes a body 12 having a first end 14 and second end 16. The body 12, as seen in FIG. 1, has a general length and a width and can be made to a variety of length and width dimensions to comfortably fit user's of any size and shape, including large adults and children. Alternatively, it is also contemplated that the body 12 is circular in shape and therefore not have any ends at all, as seen in FIG. 4.

The body 12, as seen in FIG. 1, includes a first layer 18, and a second layer 20. One of the first and second layers, 18 and 20, or alternatively, both layers are manufactured from any variety of elastomeric materials including elastic and rubber, etc., and each of the first and second layers, 18 and 20 may include the same or different elastomeric materials, in any combination thereof. It is also contemplated that the body 12 can include only one layer of elastomeric material or, alternatively, more than two layers of elastomeric or non-elastomeric material, in combination.

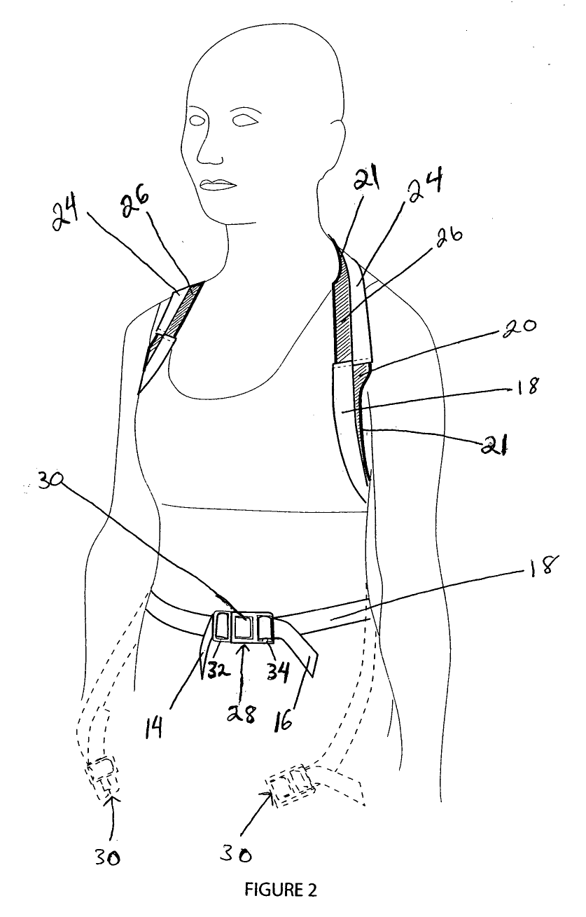

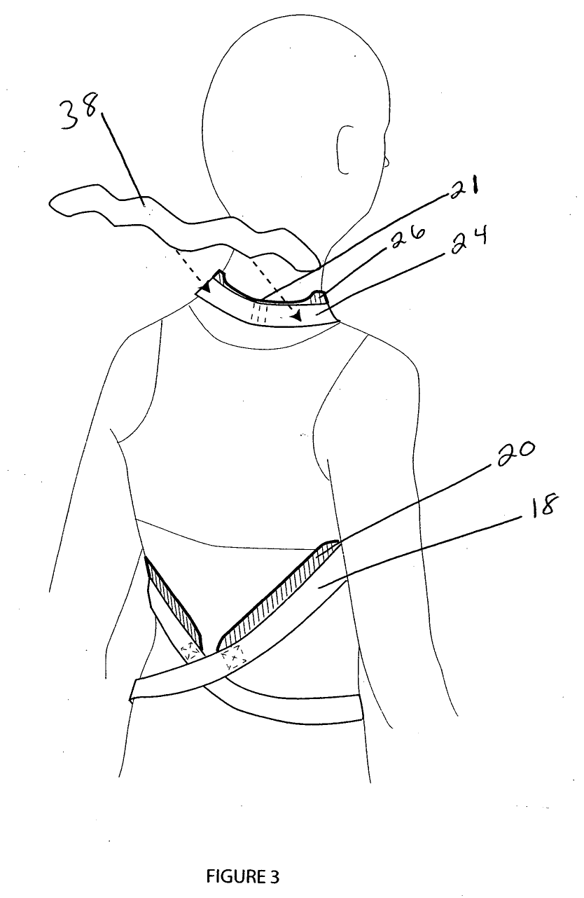

A positioning mechanism 22, seen in FIG. 1, is coupled to the body 12 for facilitating the proper alignment and tensioning of the body on a user, as seen in FIGS. 2 and 3. The positioning mechanism also includes a first layer 24 and a second layer 26, as seen in FIG. 1. One of the first and second layers, 24 and 26, or alternatively, both layers, 24 and 26 are manufactured from any variety of elastomeric materials including elastic and rubber, etc. and each of the first and second layers, 24 and 26 may include the same or different elastomeric materials, in any combination thereof. Similar to the body 12, it is also contemplated that the positioning mechanism 22 can include only one layer of elastomeric material or, alternatively, more than two layers of elastomeric or non-elastomeric material, in combination.

As is also seen in FIG. 1, second layer 26 of the positioning mechanism 22 is integral with second layer 20 of the body 12, and first layer 24 of the positioning mechanism 22 and first layer 18 of the body 12 are sewn together. Layers 24 and 26 of the positioning mechanism 22 can be secured to layers 18 and 20 of the body 12, respectively, by any means known in the art, for example, sewing, riveting, and gluing, etc., and each may alternatively be integral with the body 12, or any combination thereof.

In use, the body 12 wraps around the back of the neck, over both shoulders and around the back of the user, as seen in FIGS. 2-4, and further around the stomach of the user, as seen in FIGS. 2 and 3. The positioning mechanism 22 rests on the back of the neck of the user, as seen in FIGS. 2-4, training the user to externally rotate his/her shoulders back bringing the shoulder blades inferior and medial toward a longitudinal midline of the user and holding up his/her head. Wearing of the posture band 10 of the present invention will train the user's muscles to achieve and maintain proper posture, as described above, and create a memory in the user's muscles to maintain this proper posture even when the user is not wearing the posture band.

The Body 12 further incorporates one or more recesses 21 cut into the width of the second layer 20, as seen in FIG. 1, to facilitate the proper positioning of the body on the user as seen in FIGS. 2 and 3. As seen in FIG. 1, two recesses 21 are cut into the width of layer 20, one on either side of the coupled positioning mechanism 22.

Further, the positioning mechanism 22 includes a recess 21 cut into the width of second layer 26, as seen in FIG. 1, also to facilitate the proper positioning of the positioning mechanism 22 on the back of neck of the user, as seen in FIGS. 2 and 3. It is also contemplated that the recesses 21 can extend into first layers 18 and 24, respectively, or alternatively, a recess may be cut into first layers 18 and 24, instead of second layers 20 and 26, respectively, at points which would facilitate the positioning of the body 12 and positioning mechanism 22 on the user.

Second layer 20 of body 12 and second layer 26 of positioning mechanism 22 also have a wider width than first layers 18 and 24, respectively, as seen in FIG. 1. The wider width of second layers 20 and 26 facilitates the varied tensioning and the correct positioning and fit of the band on the user, as seen in FIGS. 2 and 3, and second layer 20 also provides support for the lower back of the user while in use, as seen in FIG. 3. The reverse width configuration is alternatively contemplated, with first layers 18 and 24 being wider than second layers 20 and 26, respectively.

The first layer 24 of positioning mechanism 22 also has a longer length than second layer 26, as seen in FIG. 1, providing a variable tension along the positioning mechanism as it is stretched while in use. The first layer 24 also includes an elastomeric material which is more difficult to stretch as compared to second layer 26, also providing a variable tension along the positioning mechanism as it is stretched while in use. Additionally, a variety of stitching lines 19 may also be included along the length of the body 12, as seen in FIG. 1, to provide a specific amount of tension directed along specific sections of the body 12 as it is stretched while in use.

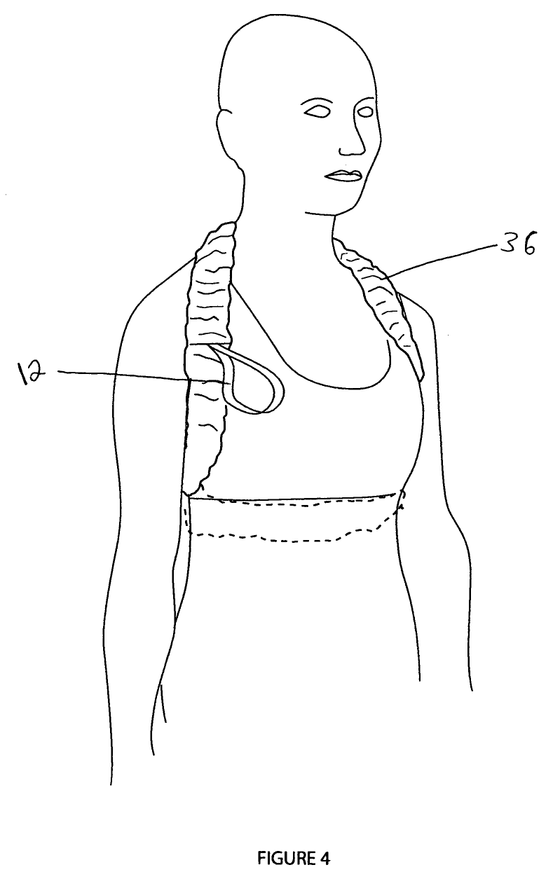

As seen in FIG. 2, a buckle 28 is coupled to both first end 14 and second end 16. The buckle 28 includes a hooking mechanism 30 and a post positioned on each of a first side 32 and a second side 34 of the hooking mechanism 30, as seen in FIG. 5 As seen in FIG. 2, end 14 is threaded around post 33 on first side 32 and end 16 is threaded around post 35 on second side 34. The hooking mechanism 30 can be easily opened and closed, as shown in FIG. 2, to easily release the tension of the band and/or remove it from the user.

Unlike standard buckles known in the art however, the unique design of the buckle mechanism 30 of the present invention allows both ends 14 and 16 to provide means for adjusting the tensioning of the body 12 while in use. Either end 14 or 16 can be pulled tighter or loosened up to facilitate the correct fit of the body 12 on the user while the user is wearing the band. This unique design allows a user to continuously wear the posture band of the present invention as a user transitions from resting to playing and/or working because the band can to be quickly and simply loosened or tightened while worn by the user, thus adjusting the specific amount of tension exhibited by the band when worn during various activities.

As seen in FIGS. 1 and 4, a covering 36 incorporates a substantial portion of the body 12 therein. A stiffening plate 40 attached to first end 32 and a stiffening plate 42 attached to second end 34 are coupled to the covering 36 by any means known in the art, such as sewing, gluing, riveting, etc. Omitting ends 14 and 16 of the body 12, as well as the buckle 28, from incorporation into the covering 36 however, will further facilitate the ease with which a user can adjust the band while in use, as described above.

The covering 36 is manufactured from a wearable material such as silk, cotton, polypropylene, etc., or any combination thereof. The polypropylene material is particularly useful if the posture band 10 is to be worn during exercising. The covering 36 provides a more comfortable feel for the user and is also fashionable and can include a stripe or poka-dot pattern, allowing the user to wear the band on top of as well as underneath their clothing.

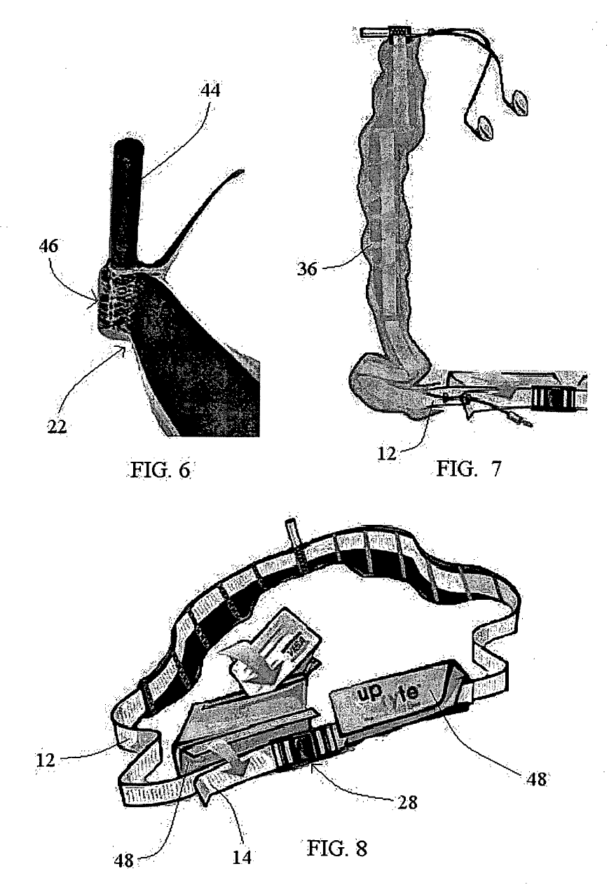

The covering 36 may also contain openings for providing access to the inside surrounding the body 12 contained therein, whereby cords for electronic devices such portable digital music players, DMPs, DVDs, IPods™, MP3 players, and the like, worn on the user, can be threaded through and contained largely within the cover 36, as seen in FIG. 7. Thus the covering 36 may further facilitate use of the body 12 as being capable of incorporating use with such portable music players. The ability to contain and conceal cords of the type described above enhances the users comfort when wearing such devices along with the posture band 10.

A flap 48 may also be coupled to the posture band 10, either removably or non-removably, by any means known in the art, including Velcro, sewing, gluing, riveting etc., to conceal the buckle 28 and ends 14 and 16 for a more aesthetic look when in use. The flap 48 structure is generally a rectangular shape and secures around the body 12 as described for use as a cover or holding structure covering the buckle, ends of the posture band body and for securing personal accessories and effects, etc. The flap 48 can be any variety of shapes and sizes and made of any variety of materials such as polypropylene or similar material having enough structure to hold identification cards and/or a money compartment etc. and yet flexible enough to be comfortably worn by a user, as seen in FIG. 8. The flap 48 is also an excellent location for personal identification markings, or advertising, or other personal effects.

A loop of the body 12 protrudes from the covering, as seen in FIG. 4, provides another means for adjusting the tensioning of the body 12 while in use. As seen in FIG. 4, the body 12 can be circular in shape and tightening or loosening the band while in use can be accomplished by pulling on or loosening up the exposed body 12. Additionally, a means for securing the desired tensioning of the band, once achieved, may include a button and slot arrangement located on the exposed loop of the body 12 to adjust and secure the correct size and fit of the band on the user.

The posture band 10 further includes a first semi-rigid insert 38 which is positioned adjacent the body 12 to enhance the effect of the positioning mechanism 22, as seen in FIG. 3. The insert 38 can be any variety of shapes and sizes, and has a generally longer length then its width, similar to the positioning mechanism 22, as seen in FIG. 3. The insert 38 is manufactured from any variety of semi-rigid material such as plastic or resin, etc., or any combination thereof.

As seen in FIG. 3, the insert 38 is located adjacent the positioning mechanism 22 and may be coupled either removably or non-removably, to the positioning mechanism 22 by any means known in the art. The first insert 38 can be at least partially contained within or may be provided as a profile or template for the covering 36 providing an alternative means for holding the insert 38 in place and providing a comfortable feel for the user. Further, a second semi-rigid insert may be coupled either removably or non-removably to the first semi-rigid insert 38 by riveting, etc. or any other securing means known in the art. The second semi-rigid insert may be manufactured from the same materials and may be generally the same shape and size as insert 38, or alternatively, a somewhat smaller size but generally the same shape as insert 38 further enhancing the effect of the positioning mechanism 22 while in use.

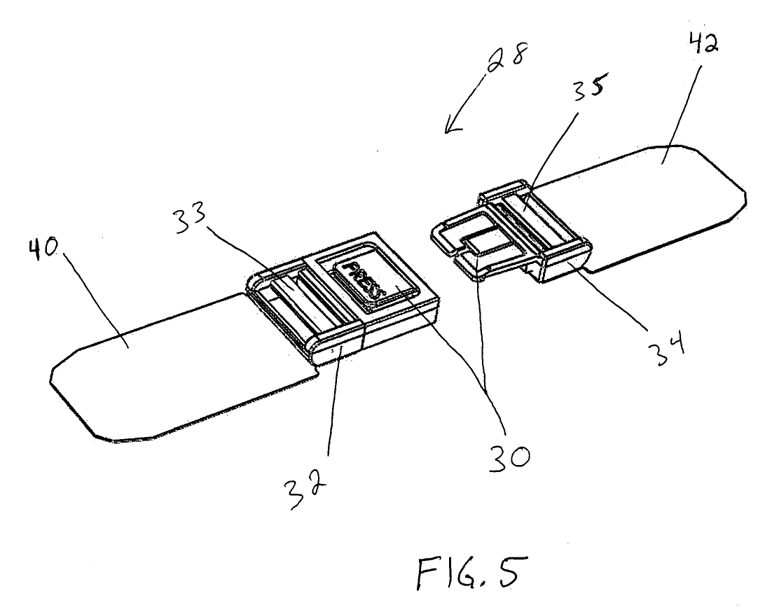

The posture band 10 further includes a foam insert 44 located adjacent the positioning mechanism 22 and may be removably coupled to the positioning mechanism 22 by any means known in the art, such as Velcro sewing, gluing, riveting, etc. As seen in FIG. 6, the foam insert 44 is generally small, about 1½ inches tall and is removably inserted into a loop portion 46 of the positioning mechanism 22. It is also contemplated that the foam or like material can be any variety of shapes and sizes and can be permanently attached to the body 12 or positioning mechanism 22. When in use, the foam portion 44 provides the user with a gentle tactile reminder to the lift up his/her head.

A method for improving a user's posture includes the steps of providing a posture band having a body made of an elastomeric material, coupling a positioning mechanism to the body and wrapping the body around the back of the neck, over both shoulders and around the back of the user. Adjusting the positioning mechanism of the band such that the positioning mechanism rests on the back of the neck of the user trains the user to roll his/her shoulders back bringing the shoulder blades together and holding up his/her head. Additionally, cutting one or more recesses into a width of the body facilitates the proper positioning of the body on the user.

Providing a first layer and a second layer of elastomeric material coupled together as the positioning mechanism facilitates the proper tensioning of the body on the user, and lengthening only one of the first or second layers of the positioning mechanism provides for a varying tension along the positioning mechanism as it is stretched while in use. Further coupling a semi-rigid insert to the body enhances the effect of the postioning mechanism

Providing a buckle coupled to both a first end and a second end of the body provides a means for adjusting the tension of the body while in use from either of the first or second ends. Tightening or loosening either end of the body facilitates the correct fit on the user allowing the user to continuously wear the posture band of the present invention while transitioning from resting to playing and/or working thus simply and efficiently adjusting the specific amount of tension exhibited by the band when worn during various activities.

Incorporating a substantial portion of the body within a covering provides a more comfortable and fashionable feel for the user, however omitting the buckle and first and second ends of the body from the covering will still allow for the easy and efficient adjusting of the tensioning of body while worn by the user, as described above.

The good health and flexibility of a person's spine can greatly enhance that persons' overall health. Likewise a person's posture and alignment of their spine significantly influences a person's long term health and well being. When the posture band 10 of the present invention is worn it is believed to stimulate the proprioceptive function of the mechanoreceptors of the muscles, so that the tension of the band (which is pulling in the direction of the less active postural muscles (primarily but not limited to the middle and lower trapezius and serratus anterior muscles) increases the activity of the less active muscles, and assists the overused/ over active muscles (primarily but not limited to the pectoralis major, pectoralis minor, upper trapezius and levator scapulae muscles) to relax by having the tension of the band pulling in the opposite direction of the contraction vector of the overactive/ overused muscles and likely also stimulating a reciprocal inhibition effect.

From the foregoing, it can be seen that there has been provided a unique posture band which is comfortable to wear and easily positioned on a user to train and maintain proper posture while engaging in any variety of activities. While a particular embodiment of the present invention has been shown and described, it will be obvious to those skilled in the art that changes and modifications may be made without departing from the invention in its broader aspects. Therefore, the aim in the appended claims is to cover all such changes and modifications as fall within the true spirit and scope of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A posture band, comprising:
an elastomeric body; and
a positioning mechanism coupled to the body for facilitating the proper alignment and tensioning of the body on a user wherein the positioning mechanism rests on the back of the neck of the user training the user to roll his/her shoulders back bringing the shoulder blades together and holding up his/her head.

2. The posture band according to claim 1, further comprising one or more recesses cut into a width of the body for facilitating the proper positioning of the body on the user.

3. The posture band according to claim 1, wherein the positioning mechanism comprises a first and second layer of elastomeric material coupled together.

4. The posture band according to claim 1, wherein the elastomeric body receives a flap structure for securing personal effects.

5. The posture band according to claim 4, wherein the first layer of the positioning mechanism has a longer length than the second layer providing a variable tension along the positioning mechanism as it is stretched while in use.

6. The posture band according to claim 5, wherein the positioning mechanism is integral with the body and at least a portion of the body further includes a first and second layer of elastomeric material coupled together.

7. The posture band according to claim 5, wherein one of the first or second layers of the positioning mechanism has a wider width than the other of the first or second layers of the positioning mechanism.

8. The posture band according to claim 1, further comprising a first semi-rigid insert laying adjacent the body to enhance the effect of the positioning mechanism.

9. The posture band according to claim 8, further comprising a second semi-rigid insert coupled to the first semi-rigid insert.

10. The posture band according to claim 1, wherein the body includes a first end and second end and further comprises a buckle coupled to both first and second ends to facilitate the correct fit of the body on the user and provide a means for adjusting the tensioning of the body while in use from either of the first or second ends.

11. The posture band according to claim 8, further comprising a covering capable of incorporating a substantial portion of the body therein, the covering being made of at least one of the following: silk, cotton or polypropylene material.

12. The posture band according to claim 11, wherein the first semi-rigid insert is at least partially contained within the covering.

13. The posture band according to claim 11, wherein the covering capable of incorporating use with a portable music player.

14. A method for improving a user's posture, comprising the steps of:
providing a posture band having a body made of an elastomeric material, and a positioning mechanism coupled to the body;
wrapping the body of the band around the back of the neck, over both shoulders and around the back of the user;
adjusting the positioning mechanism of the band such that the positioning mechanism rests on the back of the neck of the user;
training the user to roll his/her shoulders back bringing the shoulder blades together and holding up his/her head.

15. The method of claim 14, further comprising the step of cutting one or more recesses into a width of the body for facilitating the proper positioning of the body on the user.

16. The method of claim 15, wherein the positioning mechanism further provides a first and second layer of elastomeric material coupled together for facilitating the proper tensioning of the body on the user.

17. The method of claim 16, further providing the step of lengthening only one of the first or second layers of the positioning mechanism providing for a varying tension as the positioning mechanism is stretched while in use.

18. The method of claim 14, wherein the body further provides first and second ends and further comprising the step of coupling a buckle to the body for adjusting the tension of the body while in use from either of the first or second ends.

19. The method of claim 14, further comprising the step of coupling a semi-rigid insert to the body to enhance the effect of the positioning mechanism.

20. The method of claim 19, further comprising the step of incorporating a substantial portion of the body within a covering.
